(19) United States
(12) Patent Application Publication
(10) Pub. No.: US 2026/0062663 A1
(43) Pub. Date: Mar. 5, 2026

(54) Submersible Sensor Capsule and System

(71) Applicant: University College Cork, National University Ireland, Cork, Cork (IE)

(72) Inventors: Patrick Sugrue, Killarney (IE); Paul Galvin, Macroom (IE); Carlo Webster, Cork (IE); Colm Barrett, Cork (IE); Yuan Hu, Carrigaline, Co. Cork (IE); Miomir Todorovic, Cork (IE)

(21) Appl. No.: 18/997,701

(22) PCT Filed: Jul. 21, 2023
(86) PCT No.: PCT/EP2023/070382

§ 371 Date: Jan. 22, 2025

(30) Foreign Application Priority Data
Jul. 22, 2022 (GB) 2210738.7

(51) Int. Cl. C12M 1/34

(52) U.S. Cl. CPC C12M 41/26; C12M 41/32; C12M 41/34

(57) ABSTRACT

A submersible sensor capsule (10) is provided for monitoring one or more parameters of a liquid body in which the capsule is submersed. The capsule comprises one or more sensors (131) configured to measure one or more parameters of the liquid in which the capsule is submersed. The capsule further comprises an electronics assembly comprising a sensor circuit (13) in electrical communication with the one or more sensors and a wireless communications system (11) configured to transmit measured parameter information to an external device. The capsule is configured to isolate the electronics assembly from the liquid and permit liquid to contact at least a portion of the one or more sensors and the capsule is configured to float at a depth within the liquid body.

### FIELD OF INVENTION

This invention relates to a submersible sensor and system for sensing one or more parameters of a liquid. Particularly, but not exclusively, the invention relates to a submersible sensor capsule for measuring one or more parameters of a liquid such as in a bioreactor.

### BACKGROUND

A bioprocess is an industrial unit operation which involves the maximized growth of (typically a large population of) living microbial or animal cells in order to induce the biochemical conversion of a mixture of nutrients into a number of commercially important products such as biologics (e.g. antibodies, vaccines, medicines etc.), bio-fuels, biopolymers, specialty chemicals, fermented foods, etc. Bioprocesses are typically carried out in a bioreactor: a vessel for holding a liquid medium in which living cells are suspended (a cell suspension or bio-suspension) under controlled conditions. Unlike the manufacturing processes of chemicals, bioprocesses are highly sensitive to environmental conditions and even the smallest of changes in the process conditions can influence process yield. In particular, the cells are sensitive to various process parameters/variables associated with the liquid within the bioreactor, such as pH level, temperature, and dissolved oxygen level. As such, to ensure there is consistency between batches of products being produced using the living cells, the liquid parameters within the bioreactor are continuously monitored and controlled by the manufacturer.

Sensors are used to monitor the process parameters/variables within the bioreactor. Current sensor technology relies on fixed, single position sensor probes. These probes are disposed within the bioreactor (typically on the end of a rod) and provide parameter measurements for the liquid passing over the probe. Bioreactors can be large, and parameters may vary throughout the liquid. For example, some regions of the bioreactor may have higher temperatures than other regions. As such, a fixed, single position probe does not enable manufacturers to accurately monitor all the contents of the bioreactor.

The Fraunhofer Institute for Electronic Nano Systems has developed the “Sens-o-Spehere”. This is a small spherical sensor which is submersible within a bioreactor to obtain temperature measurements which can subsequently be viewed by the user. However, there are other parameters which need to be monitored within a bioreactor, and it can be cumbersome for a manufacturer to repeatedly deploy and retrieve the sens-o-sphere to monitor the temperature.

The present invention has been devised with the foregoing in mind.

### SUMMARY OF INVENTION

According to a first aspect of the invention, there is provided a submersible sensor capsule for monitoring one or more parameters of a liquid body in which the capsule is submersed. The capsule may comprise one or more sensors configured to measure one or more parameters of the liquid body. The capsule may comprise an electronics assembly or system. The electronics assembly may comprise a sensor circuit in communication with the one or more sensors. The electronics assembly may comprise a wireless communications system. The electronics system may comprise a battery for powering the capsule electronics. The communications system may be configured to transmit measured parameter information to an external device. The measured parameter information is generated, at least in part, by the one or more sensors. The capsule may be configured to isolate the electronics assembly from the liquid body. The capsule may be configured to permit liquid to contact the one or more sensors. The one or more sensors may be arranged with a surface in contact the liquid when the capsule is submerged. The capsule may be configured to float or be suspended by the surrounding liquid at a specified depth within the liquid body. The capsule may be configured to measure one or more parameters within a bioreactor, and the liquid body may be the contents of the bioreactor. The capsule may have a pre-set or adjustable buoyancy so as to float or be suspended by the surrounding liquid at a specified depth within the liquid body.

Herein, the terms “float”, “suspend” and “submerge” and “submersed” are used interchangeably. “Float” is not restricted to meaning “float at the surface”. For example, a capsule “floating” at a fixed depth in a liquid may be described as being “suspended” or submersed within a liquid.

“Bioreactors” are referred to throughout this specification. However, the invention is not limited for use with bioreactors, and the capsule presented herein can be used to measure parameters in any liquid body. A bioreactor is merely an example of where such a capsule may be used.

Having a submersible sensor capsule that remotely obtains and transmits liquid parameter information enables a user to monitor a liquid without having to interfere with the liquid directly. This reduces the chance of causing contamination. It also reduces the overall disruption to the liquid. This is beneficial, for example, in bioreactors, where interference or contamination can lead to damaged produce.

Having a buoyant submersible sensor capsule that is mobile and can float and move about within the liquid body, rather than a fixed sensor, enables parameter measurements to be taken throughout various regions of the liquid body. This enables a user to monitor the entire liquid body, rather than specific regions in the vicinity of a fixed sensor. This enables the user to determine any heterogeneity in critical parameters between regions and modulate/change conditions accordingly. For example, in a bioreactor, a user can modulate the process conditions by adjusting impeller motion, or through the addition of chemical or biochemical ingredients accordingly to ensure the entire liquid body is suitably controlled. Similar monitoring and process control can be applied to other controlled liquid bodies.

Isolating the electronics from the liquid prevents damage to the electronics, e.g. by preventing short circuits. Further, the isolation prevents the electronics from interfering with and contaminating the liquid. The only electronic components that can contact the liquid are the parameter sensors. The rest of the electronics is isolated from the liquid.

Parameter information includes measured parameter values, and may further include a measurement time stamp for the value, a determined position of the capsule associated with measured parameter value and/or location information for determining a position of the capsule associated with measured parameter value.

The communications system may be configured to transmit measured parameter information in real time to an external device. In this context, “real time” means transmission without delay, such as transmission when a measurement is taken. Alternatively, the capsule may be configured to store parameter information measured over a period of time and transmit a plurality of measurements at once. Parameter information may be time stamped.

The communications system may be configured to transmit measured parameter information at a certain transmit frequency or at times or time intervals according to a predefined schedule. The parameter information may be sent to the external device wirelessly after each measurement/sample.

Alternatively or additionally, the communications system may be configured to selectively transmit measured parameter information to an external device in response to a trigger signal received from the external device and/or in response to a detected trigger event. This assists with power saving, as sending the same parameter information continuously, rather than only when there is a sufficient change in parameter values, would consume more power.

A trigger event may comprise one or more of: a threshold change in one or more of the measured parameters, a threshold rate of change in one or more of the measured parameters, and a detection of a parameter.

The trigger event and/or the thresholds may be adjusted and set/defined by a control signal received from the external device. This allows the resolution of the change in any parameter to be set by the controller and changed during operation. Alternatively or additionally, the trigger events and/or the thresholds may be adjusted according to a predefined schedule.

Where the communication system transmits the measured parameter information at a transmit frequency, the communication system may be configured to alter the transmit frequency in response to one or more of: a change in the operating mode of the capsule (e.g. a sleep mode, wake/active mode, high or low resolution mode), a trigger signal received from the external device, and a detected trigger event (as above). The trigger signal may comprise a command signal to change the transmit frequency directly or change an operating mode. The transmit frequency may be adjusted to minimise energy usage and preserve power.

The electronics assembly may comprise a processing circuit configured to analyse the measured parameter information to detect a trigger event. For example, the processing circuit may use EDGE AI algorithms to perform preliminary analysis and optimise the power efficiency by adjusting the communication frequency based on detection of parameter measurements outside of set tolerances.

The one or more sensors may measure one or more parameters at a sample or measurement frequency. This sample frequency may be adjustable. In one embodiment, the sample frequency may be adjusted in response to a command/control signal received from the external device. The processing circuit may be configured to sample the output of the one or more sensors at a measurement frequency. The processing circuit may be configured to alter the measurement frequency in response to one or more of: a change in the operating mode of the capsule, a trigger signal received from the external device, and a detected trigger event. The sample frequency may be adjusted to minimise energy usage and preserve power. The sample frequency may also be adjusted according to software stored in a memory of the capsule.

By transmitting parameter information in real time or in response to certain changes in parameter values, a user who is monitoring the liquid can react quickly to any undesirable changes in parameter values and has sufficient opportunity to interfere with the liquid before irreversible damage is caused. For example, the user may be able reduce the heat being applied to a bioreactor if the temperature of the liquid is too high.

The capsule may comprise a memory configured to store the measured parameter information. The communications system may be configured to transmit measured parameter information from the memory to an external device. The communications system may be configured to transmit measured parameter information from the memory to an external device periodically.

The processing circuit may be configured to analyse the measured parameter information immediately after the measured parameter information is obtained. The processing circuit may be configured to analyse the measured parameter information by retrieving it from the memory. The processing circuit may be configured to determine whether measured parameter information shows that the measured parameter values fall outside of a predetermined range. The processing circuit may be configured to determine whether the measured parameter information indicates a change in the measured parameter values. The communications system may be configured to selectively transmit measured parameter information based on the analysis from the processing circuit.

By only transmitting specific information, the capsule can conserve energy. For example, the capsule may only transmit parameter information when action needs to be taken by a user, rather than continuously. This enables the capsule to be deployed for longer without replacing the battery. Further, it enables the capsule to use a smaller battery.

The battery may be rechargeable, and optionally wirelessly rechargeable. This may permit in situ charging of the capsule within the liquid body.

The capsule may comprise a sensor bay region in which the one or more sensors are positioned and/or mounted. The capsule may further comprise one or more, or a plurality of, side openings to allow liquid to enter the sensor bay region to contact the one or more sensors.

The capsule may have a modular construction, comprising a plurality of removably connectable and/or interchangeable sections.

The capsule may comprise a circuitry section and a sensing section. The circuitry section may be removably connected to the sensing section along a longitudinal axis of the capsule. The circuitry region may house the electronics assembly. The connected circuitry section and sensing section may define an enclosed space within the circuitry section that houses the electronics assembly. The sensing section may at least partially define the sensor bay region. The enclosed space of the circuitry section may be fluidly isolated from liquid in the sensor bay region. The sensing section may comprise the one or more or a plurality of openings configured to enable liquid to flow into the capsule to contact the one or more sensors.

A plurality of openings may allow the liquid to flow in and out of the sensor bay region without disrupting the liquid significantly.

The capsule may comprise a fluidic actuator. The fluidic actuator may be a micropump. The fluidic actuator may be configured to convey liquid into and/or through the sensor bay region of the capsule to the one or more sensors.

The capsule may comprise an aperture between the circuitry section and the sensor bay region to permit liquid in the sensor bay section to contact the one or more sensors. The aperture may be in a longitudinal end of the sensing section adjacent the circuitry section. The one or more sensors may be positioned or mounted over or within the aperture. The one or more sensors may be positioned such that it is configured to contact liquid within the sensor bay section. The one or more sensors may be positioned such that it is configured to seal the aperture to prevent liquid entering the circuitry section.

Using the one or more sensors to seal the aperture simplifies the design of the capsule and reduces the total number of components required to construct the capsule.

The capsule may further comprise a ballast control section. The ballast control section may be configured to control the depth at which the capsule floats within the liquid. The ballast control section may be configured to control the orientation of the capsule within the liquid.

Enabling the capsule to be submersed at different depths enables parameters to be measured throughout the entire liquid body, rather than, for example, just at the surface. In a bioreactor, or other vessel housing a liquid body, the parameters of the liquid body may vary according to depth, and so the user needs to be able check the parameters at different depths.

By controlling the orientation of the capsule, a user can ensure that the communications system is pointing in a desired direction, for example facing upwards. This enables the communications system to more easily transmit information to the external device. The communications system may transmit information to the external device via a floating gateway device (also referred to below as a surface repeater), which can include communications modules optimised for multiple media, such as air and liquid. Controlling the orientation of the capsule and the communications system enables the communications system to be oriented towards, and more easily transmit, information to the floating gateway device. Controlling capsule orientation can also facilitate optimal communication between a plurality/mesh of capsules submersed in the same liquid body.

The ballast control section, circuitry section and sensing section may be removably connected to form the capsule. Additional sections may be added due to the modular construction of the capsule. The ballast control section may be located at a longitudinal end of the capsule. The sensing section may be connected between the ballast control section and the circuitry section. The circuitry section may be connected to a first end of the sensing section. The ballast control section may be connected to a second, opposing end of the sensing section.

The connections between the sections may be a threaded connection. The connections may be sealed using O-rings.

Threaded connections are beneficial since they provide a secure but releasable connection. A user may wish to remove sections of the capsule for repair/replacement, so it is useful to have releasable connection.

The communications system may be configured to receive signals from an external device. The signals may be control signals and/or trigger signals. The signals may comprise instructions or commands. The instructions may be to set or change the measurement frequency. The signals may provide software updates for processor in the capsule.

For example, if the capsule has low battery power left, the instructions can instruct the capsule to take parameter measurements with a lower frequency. As another example, if the parameters need to be tightly controlled, the instructions can instruct the capsule to obtain parameter measurements with a higher frequency, or in response to crossing smaller thresholds.

The electronics assembly may comprise a layered stack of electrical components. The circuitry section houses the layered stack. The communications system and the sensor circuit may be disposed on separate layers of the stack. The electronics system may comprise additional electrical components. The additional electronic components may form part of the layered stack of electrical components. The additional components may comprise one or more of: a battery, a control system, a memory, a processor, an analogue to digital converter (ADC), a digital to analogue converter (DAC), and an analogue front-end adapter.

The communications system may be disposed at a first end of the layered stack and the one or more sensors may disposed at a second of the layered stack, opposite the first end. The second end of the layered stack may be proximate the sensor bay section and the first end may be distal to the sensor bay section. The capsule may be configured (using the ballast control section), when submersed in liquid, to be oriented such that the first end is above the second end. In this way, the communications system may be disposed at the top of the layered stack in use (with respect to the orientation of the submersed capsule).

Positioning the communications system at the top of the layered stack enables the height of the communications system to be maximised which assists with sending signals out of the liquid to the external device.

The one or more sensors may be configured to measure one or more of: pH level, temperature, glucose, lactate, and dissolved oxygen. The sensor may be configured to measure other parameters in the liquid body. The sensor may be configured to measure parameters in the liquid body using electrochemical, optical or other sensing modalities.

The electronics assembly may comprise a processing circuit configured to determine a location or position of the capsule within the liquid body based on signals received from a plurality of external locator devices, optionally or preferably using triangulation or multilateration. The capsule communications system may be configured to send the determined position information to the external control device. The transmitted parameter information may includes the capsule position at the parameter measurement time.

The capsule may comprise one or more positional sensors configured to measure a position of the capsule within the liquid. The positional sensors generate sensor data containing position information of the capsule within the liquid. The capsule communications system may be configured to send the sensor data to the external device. The one or more position sensors comprise an accelerometer and/or an inertial measurement unit. The processing circuit may be configured to determine a location or position of the capsule within the liquid body based on signals received from a plurality of external locator devices and the sensor data.

By determining parameters at different locations/positions of the liquid specifically, the user can identify problem areas within the liquid body. This may enable the user to identify faults with a liquid process or bioreactor.

The capsule may comprise a plurality of sensors for measuring different parameters of the liquid. Each sensor may be configured to measure a different parameter. Alternatively, each sensor may be configured to measure a plurality of parameters. The plurality of sensors may be located in a single sensor bay section.

Alternatively, the capsule may comprise a plurality of sensor bay regions, whereby each sensor bay region comprising one or more of the plurality of sensors. The plurality of sensor bay regions may be defined by the sensing section. Alternatively, the capsule may comprises multiple connectable sensing sections that connect to each other and/or the circuitry section, each sensing section defining a separate one of the sensor bay regions.

Having sensors within different sensor bay regions/sections prevents sensors from interfering with or contaminating each other (e.g., such as for glucose and lactate, where the primary measurement is for hydrogen peroxide as a by-product of the reaction of the target metabolite with either the glucose oxidase or lactate oxidase immobilised on the sensor). Further, the use of multiple sensor bay sections and/or sensors enables the capsule to measure more parameters.

The communications system may be configured to generate radio frequency (RF) signals. The communications system may be configured to generate acoustic signals. The communications system may be configured to generate ultrasound signals.

Where the communication system generated RF signals, it comprises an antenna. The antenna may be attached to a layer of the stack. Alternatively, the antenna may be formed/printed on an interior surface of the circuity section in a custom pattern. This provides a larger antenna area and better control the EM radiation pattern. For example, an antenna can be formed which directs the main lobe of the EM radiation pattern upwards (in use when the capsule is submerged) towards a receiver device which may be located at or near the top of the liquid containing vessel (e.g. a controller antenna can be inserted into a bioreactor port which is usually on the top of traditional bioreactors).

With a modular construction, components of the capsule can be replaced, added, or removed. For example, at least one of the one or more sensors may be replaced in order to measure different parameters. As another example, the communications system may be replaced. This enables the capsule to be used in a variety of environments. If the capsule is intended to be submerged in a stainless-steel bioreactor, then an ultrasound wireless communications system may be used, whilst for a glass container, an RF wireless communications system may be used. An RF module (one type of wireless communications system) can be substituted by an alternative wireless communications system to avail of alternative communications protocols, such as ultrasound, acoustic or optical.

The modularity may be achieved through the layered stack of electrical components. For example, a user may remove the layered stack from the capsule to remove and add specific components from/to the layered stack. Arranging the electrical components in a layered stack enables a user to easily disassemble and reassemble the components. Modularity enables the production of bespoke capsules configured for use in a range of liquid bodies, and which are configured to measure a variety of different parameters.

The capsule may further comprise a micro or nano-bubble generator configured to clean a surface the one or more sensors in contact with the liquid. The micro or nano-bubble generator may direct generated micro or nano-bubbles over the surface of the sensor. This removes any contaminants that may build up on the sensors, a biofouling layer, and allows the sensor to be used for extended periods.

The micro or nano-bubble generator may be part of the sensor. The micro or nano-bubble generator may comprise electrodes which are part of the sensor. The micro or nano-bubble generator may be configured to generate the nano-bubbles by polarising a working electrode of the sensor. The micro or nano-bubble generator may be configured to generate the nano-bubbles by polarising a working electrode of the sensor in a hydrogen evolution region (HER).

According to a second aspect of the invention, there is provided a system for monitoring one or more parameters of a liquid. The system may comprise a submersible sensor capsule configured to float at a depth within the liquid, and a transceiver module connectable to an external control device. The sensor capsule may be the capsule of the first aspect of the invention. The capsule may comprise one or more sensors, and a capsule communications system connected to the one or more sensors. The sensor(s) may be configured to measure the one or more parameters of the liquid in which the capsule is submersed. The capsule communications system may be configured to send the measured parameter information to the transceiver module. The transceiver module is configured to receive measured parameter information transmitted by the capsule and provide the measured parameter information to a connected external control device. The system may comprise the external control device. The system may be configured to measure one or more parameters of the contents of a bioreactor. The control system may be external to the bioreactor.

The transceiver module may comprise a communications system configured to receive the measured parameter information sent by the capsule communications system. The transceiver module may be connected to the external control device via wired connection such as USB.

The transceiver module can be plugged into an existing external control device or retro-fitted to various different types of liquid vessel control systems to extend the functionality of the control system to wireless in situ monitoring of liquid parameters.

The transceiver module may comprise, or be attachable to, a probe configured to be disposed in the liquid to receive the measured parameter information from the capsule communications system.

In a steel bioreactor, or other steel container, communication signals (especially RF signals) may be trapped within the container. Using a probe which enters the liquid body ensures that the transceiver can still detect signal from the capsule.

The capsule may comprise one or more positional sensors configured to produce sensor data containing positional information of the capsule within the liquid. The capsule's communications system may be configured to send the sensor data to the transceiver module together with the measured parameter information. The system may comprise a fixed reference accelerometer and the capsule may comprise a capsule accelerometer configured to produce accelerometer information.

The system may comprise one or more locator devices configured to send locating signals to the capsule. The locating signals may contain information indicating when they were first sent and where the signals originated.

The capsule may comprise a processing circuit configured to determine a location or position of the capsule relative to the one or more locators and/or the external device based on the locating signals. The processing circuit may be configured to determine a location or position of the capsule by triangulation or multilateration of RF, acoustic, or ultrasound signals received from the fixed locator device or beacons positioned around the liquid containing vessel. The capsule's communications system may be configured to send the determined position information to the transceiver module together with the measured parameter information. Optionally or preferably, the transmitted parameter information includes the capsule position at the parameter measurement time.

The processing circuit may be configured to determine a location or position of the capsule based on the locating signals and the sensing data

Alternatively or additionally, the capsule communications system may be configured to send locating signals and/or the position sensor data to the locator devices. The locator signals may include the time of transmission. The locator device may be in communication with the external control device, and the external control device may be configured to determine the location or position of the capsule based on the received locating signals (and optionally position sensor data) and the fixed positions of the locator devices, e.g. using time of flight and/or triangulation or multilateration.

The external control device may be configured to generate a map indicating measured parameter values at one or more locations in the liquid based on the received parameter information and location information. The map can be used with closed loop algorithms to ensure optimal conditions are maintained within the liquid containing vessel.

The external control device may be or comprise a controller configured to control at least one of the parameters of the liquid based at least in part on the measured parameter information, and optionally or preferably, wherein the system is a bioreactor control system.

The external device may be configured to send control signals and/or instructions to the capsule via the transceiver module.

The capsule may be rechargeable, and comprise a rechargeable battery. The system may comprise one or more charging or docking stations configured to charge the capsule battery in situ (i.e. without removing the capsule from the liquid). The capsules may connect to the docking stations to recharge. The docking/charging station(s) may wirelessly charge the capsule via inductive charging or acoustic charging. The docking stations may float at a surface of the liquid body, or at a fixed height within the liquid body. The docking stations may be fixed to an edge of a container containing the liquid body. The docking stations may be fixed at any position within the liquid body.

The charging station may be mountable at least partially within the liquid and may comprise a means to capture a capsule within the liquid and move the capsule into proximity with the charging station for power transfer.

The system may comprise one or more surface repeaters. The surface repeaters may be configured to receive signals emitted by the capsule communications system. The surface repeaters may be configured to relay/send the received signals to the transceiver module. The surface repeaters may be configured to float or be positioned at the surface of the liquid. The surface repeaters may be configured to receive signals of a first type from the capsule (using a first type of communication technology) and send signals of a second type to the transceiver module (using a second type of communication technology). The first type of signal may be an acoustic/ultrasound signal, and the second type of signal may be an RF signal.

Using surface repeaters helps to improve signal strength, especially when the signals are being transmitted over large distances. Further, the surface repeaters may help to reduce the signal loss associated with signal reflections at the liquid-air boundary. The surface repeaters also allow the use of different signal types/communications technologies appropriate to the medium to get the signals out of the liquid to the transceiver module.

The capsule communications system may be configured to send ultrasound signals to the one or more surface repeaters. The surface repeaters may be configured to send radio frequency electromagnetic signals to the transceiver module.

Ultrasound signals may be used in the liquid body to better maintain the signal strength. RF signals may then be used exclusively for signal transmission outside of the liquid body.

The system may comprise a plurality of submersible sensor capsules. Each capsule may be configured to send measured parameter information to the control device communications system. Each of the plurality of capsules may be configured to float at different depths within the liquid. Each capsule may be fixed or positioned in a distinct region of the liquid. For example, a capsule's location may be fixed using a physical barrier which restricts where the capsule can float/move within the liquid body.

According to a third aspect of the invention, there is provided a submersible sensor capsule for monitoring one or more parameters of a liquid body in which the capsule is submersed. The capsule may comprise one or more sensors configured to measure one or more parameters of the liquid. The capsule may comprise a wireless communications system configured to transmit measured parameter information to an external device. The capsule may comprise means for generating micro or nano-bubbles configured to remove contaminants from the one or more sensors. The capsule may be configured to float at a depth within the liquid body. The micro-bubbles help remove any contaminants that may build up on the sensor(s), such as a biofouling layer, and allows the sensor(s) to be used for extended periods of time.

The means for generating nano-bubbles may generate the nano-bubbles directly on a surface of the sensor. The means for generating micro or nano-bubbles may direct generated micro or nano-bubbles over the surface of the sensor. The means for generating nano-bubbles may be or comprise an electro-chemical micro/nano-bubble generator. The means for generating nano-bubbles may be part of the sensor. The means for generating nano-bubbles may comprise electrodes which are part of the sensor.

The means for generating nano-bubbles may be configured to generate the nano-bubbles by polarising a working electrode of the sensor. The means for generating nano-bubbles may be configured to generate the nano-bubbles by polarising a working electrode of the sensor in a hydrogen evolution region (HER).

The capsule may include any features or combination or features described in connection with the capsule of the first aspect.

Optional features of any of the above aspects may be combined with the features of any other aspect, in any combination. For example, features described in connection with the capsule of the first or third aspects may have corresponding features definable with respect to the system of the second aspect, and vice versa, and these embodiments are specifically envisaged. Similarly, features described in reference to the capsule of the first aspect may be used with the capsule of the third aspect, and vice versa. Features which are described in the context or separate aspects and embodiments of the invention may be used together and/or be interchangeable wherever possible. Similarly, where features are, for brevity, described in the context of a single embodiment, those features may also be provided separately or in any suitable sub-combination.

### BRIEF DESCRIPTION OF DRAWINGS

Embodiments of the invention will now be described, by way of example only, with reference to the accompanying drawings, in which:

FIG. 1(a) and 1(b) show side and sectional views of a submersible sensor capsule according to an embodiment of the invention;

FIG. 2 shows an exploded view of the capsule of FIG. 1;

FIG. 3 shows the capsule of FIG. 1 with the circuitry section body removed;

FIG. 4(a) shows a sectional view of the capsule of FIG. 1 through line A-A;

FIG. 4(b) shows the sensor PCB secured to the first longitudinal end of the sensing section.

FIG. 5 shows a schematic block diagram of the capsule electronics system;

FIG. 6 shows an exploded view of a submersible sensor capsule according to another embodiment of the invention.

FIG. 7 shows schematic diagram of a system for monitoring one or more parameters in a liquid, according to an embodiment of the invention;

FIG. 8 shows schematic diagram of a system for monitoring one or more parameters in a liquid, according to another embodiment of the invention;

FIG. 9 shows schematic block diagram of the transceiver module and controller system of the system of FIGS. 7 and 8;

FIG. 10 illustrates s a sectional view of a wireless transceiver module of the system of FIGS. 7 and 8, according to an embodiment of the invention;

FIG. 11 shows a capsule according to another embodiment of the invention; and

FIG. 12 shows a transceiver module according to another embodiment of the invention.

Like reference numerals in different Figures may represent like elements.

### DETAILED DESCRIPTION

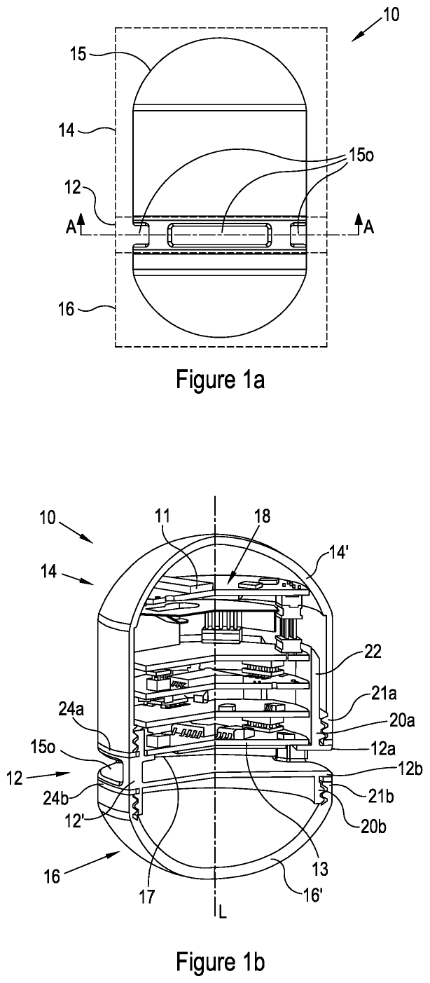

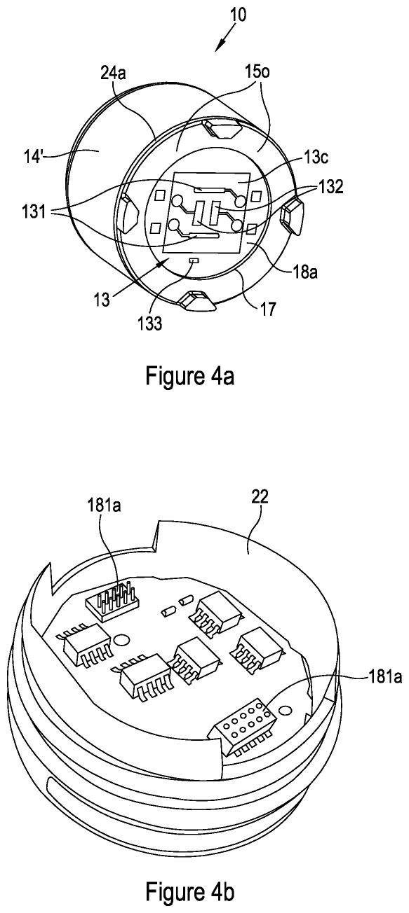

FIG. 1(a) and 1(b) show side and sectional views of a submersible sensor capsule 10 for monitoring one or more parameters of a liquid within a liquid containing vessel such as a bioreactor or other vessel used in a controlled liquid process that requires monitoring, according to an embodiment of the invention. Although described below primarily in the context of bioreactors and bioprocesses, it will be appreciated that the capsule 10 can be used to measure parameters of interest in any liquid containing vessel used in a controlled liquid process that requires monitoring. The capsule 10 comprises a substantially rigid casing 15 containing the capsule electronics assembly including a sensor circuit 13 and a communications system 11. The sensor circuit 13 is connected to one or more sensors 131 configured to measure one or more parameters of a liquid in which the capsule 10 is submersed, as described in more detail with reference to FIG. 4(a) and 4(b). The communications system 11 is configured to transmit measured parameter information to an external device such as a process controller (not shown). The casing 15 protects the internal electronic components of the capsule 10 from the liquid environment during operation. As described below, the capsule 10 is configured such that, in use, at least part of the or each sensor 131 contacts the liquid, while the rest of the capsule electronics (including the communications system 11) is isolated from the liquid.

The capsule 10 is configured to be submersed in a liquid body to a depth set by a ballast control feature (see below) and floats within the liquid media at that depth. The submersed capsule 10 is free to move about in the liquid media to measure parameters of interest throughout the liquid containing vessel. In a bioreactor these parameters may include temperature, pH and dissolved oxygen levels, to monitor mixing and aeration. Measurement data can be transmitted by the capsule 10 to an external device such as the process controller (not shown) in real time following a scheduled or requested measurement (described in more detail below) using the on-board communications system 11 to provide bioreactor heterogeneity evaluation. The process controller can then intervene early to ensure quality and yield of the bioprocess. In this way, the submersible sensor capsule 10 addresses the limitations of fixed position probes commonly used in bioreactors.

The term “float” used herein does not necessarily mean float at the surface of a liquid unless stated so, but is used generally to mean to float at a substantially fixed depth in the liquid.

The capsule 10 has a modular construction comprising a sensing section 12, a circuitry section 14, and a ballast control section 16 that connect together along a longitudinal axis L to form the assembled capsule 10. The circuitry section 14 houses the sensor circuit 13 and communications system 11 along with any other electronics. The sensing section 12 is configured to allow liquid to enter the capsule 10 and flow into contact and/or over the one or more sensors 131 for obtaining parameter measurements. The ballast control section 16 is configured to hold a weight or a fluid (liquid or gas) for controlling the depth and orientation of the capsule 10 within the liquid, as described in more detail below.

The circuitry section 12 and ballast control section 16 removably connect to respective first and second (opposite) longitudinal ends 12a, 12b of the sensing section 12 using thread 20a, 20b, 21a, 21b and o-ring 24a, 24b connection mechanisms to form sealed interior spaces within the respective circuitry and ballast control sections 14, 16. The sealed interior space of the circuitry section 14 encloses and protects the capsule electronics. The sealed interior space of the ballast control section 16 is configured to trap/hold a volume of fluid inside the capsule 10 that provides buoyancy to enable the capsule 10 to float at a certain depth within the liquid, as described in more detail below.

The removable connections enable a user to easily disassemble and reassemble the capsule 10, e.g. to access the electronics and/or exchange the fluid within the ballast control section 16. In particular, by accessing the electronics, various components can be replaced or exchanged for components suitable for particular application (e.g. communication systems, batteries, sensors), enabling a bespoke capsule to be built. Although convenient, threaded connections are not essential. Other sealable connection mechanisms can be used instead, e.g. push fit connection mechanisms, as are known in the art.

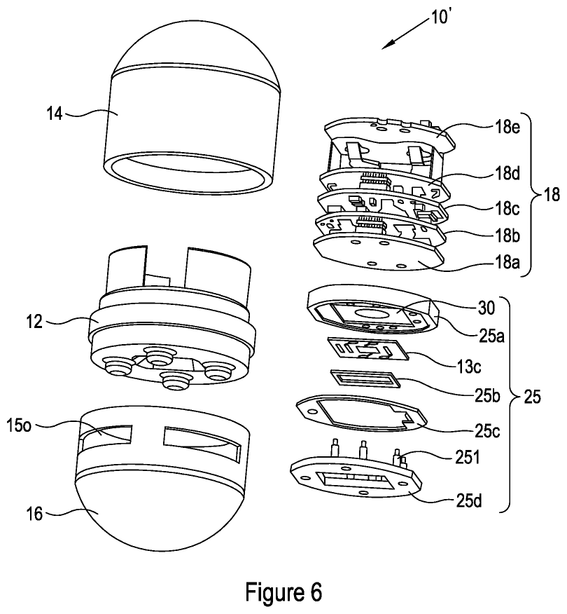

The capsule 10 comprises one or more lateral side openings 15o to allow liquid to enter a sensor bay region within the sensing section 12. The sensing section 12 comprises the side openings 15o and also an aperture 17 extending through the first longitudinal end 12a into the circuitry section 14 to allow liquid flowing through the sensor bay to contact the one or more sensors 131 for obtaining parameter measurements. In other embodiments, the ballast control section comprises the side openings 15o (see FIG. 6). As described in more detail below, the sensors or sensor circuit 13 is disposed over and/or within the aperture 17 in order to contact liquid within the sensor bay region and also to seal the aperture 17 to prevent liquid entering the interior space of the circuitry section 14.

In use, as the capsule 10 floats in the liquid media, liquid passively travels through the lateral side openings 12ointo and out of the senor bay region where it can contact and flow over the sensors 131.

The interior spaces of the circuit section 14 and ballast control section 16 are fluidly isolated from liquid in the sensor bay region. In other embodiments (not shown), the sensing section 12 may also comprise one or more fluidic actuators, such as micropumps, to actively convey liquid through the sensor bay.

The ballast control section 16 provides the mass and buoyancy to control the orientation of the capsule 10 and the depth at which the capsule 10 floats. In some embodiments the capsule 10 is configured to float at a substantially fixed depth, in which case the ballast control section 16 is configured to hold a fixed amount of solid or liquid to provide a suitable mass for the intended buoyancy. The density, mass and/or volume of ballast fluid can be chosen to provide neutral buoyancy and prevent the capsule 10 from floating to the top of the bioreactor liquid, as well as to set the floating depth. A user can disconnect and reconnect the ballast control section 16 to at least partially fill the ballast section 16 with a fluid/solid and/or change the fluid.

It will be appreciated that a suitable volume/mass of liquid or solid material in the ballast can ensure that the ballast control section 16 is heavier than the circuitry section 14 (which contains trapped air/gas) and thereby positioned below the circuitry section 14 when submersed in the liquid. This would ensure that, when the capsule 10 is submersed and floating within the liquid media, it maintains an orientation whereby the communications system 11 is positioned at or near the top of the capsule 10, at its highest possible point to aid communications with the external control system, discussed in more detail below.

In other embodiments (not shown), the ballast control section 16 may be configured to selectively/dynamically control the orientation and/or floating depth of the capsule 10 without disassembling the capsule 10. In this case, the ballast control section 16 is configured to hold a variable amount of liquid to control its mass/density and thereby adjust buoyancy, similar to how a submarine operates. In such embodiments, the ballast control section 16 comprises a source of compressed gas such as air (or any other standard atmospheric or insert gas) and a plurality of valves. Compressed gas source can be programmed to release gas into an interior space of the ballast control section to expel a volume of liquid held therein through a valve to reduce the floating depth, and a valve can be opened to allow gas to exit and liquid to enter the interior space to increase the floating depth. Alternatively or additionally, the capsule 10 can comprise a fluidic actuator such as a micro pump to convey liquid into, and out of, the ballast control section 16 to dynamically alter the buoyancy in situ within the bioreactor. In this case, the ballast fluid may comprise the liquid in which the capsule 10 is submersed.

Alternatively or additionally, the capsule 10, 10′ can be tethered to the bottom of the bioreactor vessel (not shown), e.g. using a flexible wire or cord, to set the floating depth and/or restricts the volume/region of liquid 101 in which the capsule 10, 10′ can float and move around in, allowing sensor data to be associated with that region defined by the anchor point.

Further still, the volume/region of liquid 101 in which the capsule 10, 10′ can float and move around in can be restricted by dividing the bioreactor vessel into separate regions using a mesh, grid or cage system (not shown).

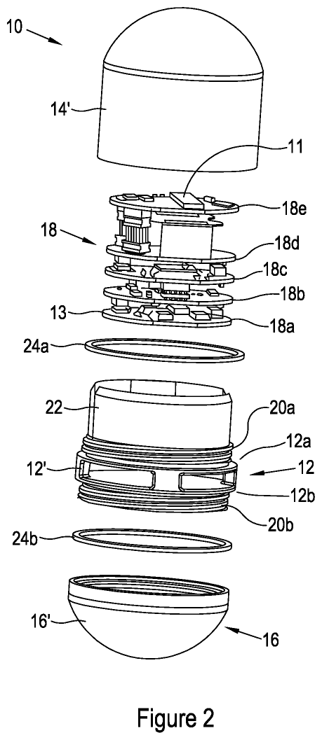

FIG. 2 shows an exploded view of the capsule 10 assembly. Each section 12, 14, 16 comprises a rigid body 12′, 14′, 16′ that connects to form the overall casing 15 of the assembled capsule 10. The sensing section body 12′ comprises a first threaded portion 20a on the first longitudinal end 12a configured to engage with a corresponding threaded portion 21a of the circuity section body 14′, and a second threaded portion 20b on the second longitudinal end 12b configured to engage with a corresponding threaded portion 21b of the ballast control section body 16′. The lateral side openings 12o, aperture 17 and sensor bay region are formed in the sensing section body 12′.

The section bodies 12′, 14′, 16′ that form the capsule casing 15 are formed of or comprise a substantially rigid plastic material (e.g. using a moulding process) to provide durability and resistance to chemical and physical damage within the bioreactor environment. In an embodiment, the plastic material is or comprises a thermoplastic, such as polycarbonate, polypropylene, polyethylene, cyclic olefin copolymer (COC), together with silicone as applicable. In particular, the casing 15 material can withstand the typical temperatures experienced in the bioreactor (e.g. 25-40 degrees C.).

The electrical components of the capsule 10 are distributed across a plurality of printed circuit boards (PCBs) 18a-18e arranged in a layered stack 18, as shown in FIG. 1(b) and 2. Each layer 18a-18e of the stack 18 comprises a PCB with one or more electrical components and one or more electrical connectors to facilitate the stacked formation. The communications system 11 is located at or near the top of the stack 18, and the sensor circuit 13 is located at the bottom of the stack 18. The layered stack 18 is described in more detail below.

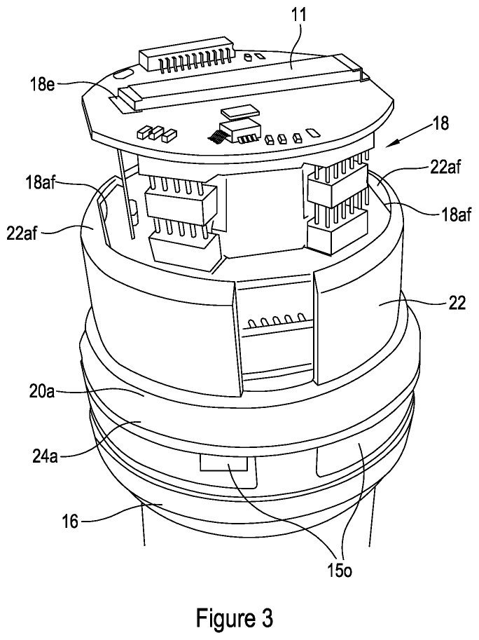

FIG. 3 shows the capsule 10 with the circuit section body 14′ removed. The sensing section body 12′comprises a sleeve portion 22 configured to receive the layered stack 18 of electrical components, as seen in FIG. 3. The sleeve 22 extends from the first longitudinal end 12a of the sensing section body 12′ and, when the capsule 10 is assembled, into the interior space of the circuitry section 14 to locate/position the layered stack 18. In the embodiment shown, the sleeve 22 comprises one or more longitudinally extending walls or projections.

FIG. 4(a) is a cross-sectional perspective view of the capsule 10 taken through line A-A in FIG. 1(a) showing the aperture 17 of the sensing section 12 and the part of the sensor circuit 13 that is exposed to liquid in the sensor bay. As seen, the bottom PCB layer 18a of the stack 18 (the sensor PCB 18a) comprising the sensor circuit 13 is positioned over and extends across the aperture 17. FIG. 4(b) shows the sensing section 12 with the sensor PCB 18a positioned over the aperture 17. The sensor PCB 18a has a diameter greater than the diameter of the aperture 17 and is positioned against the rim of the aperture 17 to provide a seal. In this example, the sensor PCB 18a is secured to the longitudinal end 12a of the sensor section 12 with a biocompatible adhesive and encapsulation polymer to create a seal around the aperture 17 and prevent liquid entering the circuitry section 14. The rest of the stack 18 connects to the sensor PCB 18a via electrical connectors 181a, which may also serve to hold it in place.

In the embodiment shown, the sleeve 22 and the layered stack 18 comprise complementary geometric alignment features 22af, 18af (in the form of lateral side flats) to aid proper alignment of the layered stack 18 and connectors 181a upon insertion (see FIG. 3). In various embodiments, the sleeve 22 may also be configured to grip/hold the stack 18 in position by frictional and/or mechanical engagement to prevent unintentional disconnection from the sensor PCB 18a. Alternatively or additionally, the layered stack 18 can be held in place against the sensing section 12 by a compressive/retaining force applied to the top of the stack 18 by the circuit section body 14 when it is securely connected to the sensing section body 12.

In the illustrated embodiment, the sensor circuit 13 comprises three sensors 131-133 attached and electrically connected to the sensor PCB 18e: two electrochemical sensors 131, 132 for measuring pH and DO levels, and a temperature sensor 133 such as a thermistor (see FIG. 4(a)). In general, the sensor 13 may comprise one or more sensors 131-133 for measuring one or more parameters of interest including but not limited to temperature, pH, DO, glucose, and lactate. The electrochemical sensors 131, 132 comprise respective sensing electrodes which are exposed to liquid in the sensing section 12 for measuring pH and DO levels. In the example shown, the electrochemical sensors 131, 132 are integrated into a sensor chip 13c which is die-attached and wire bonded to the sensor PCB 18a (not shown). In other examples, all three sensors 131-133 may be integrated into a single sensor chip 13c, or they may be separate components, depending on the use application. The sensors can be electrochemical (e.g. as illustrated in FIG. 4(a) and 4(b)), electrical (e.g. ISFETs), optical (e.g. photodiodes connected to polymers) or any other suitable sensing technology. In use, the sensor chip 13c, any bond wires and the thermistor 133 are encapsulated on the sensor PCB 18a using a bio-compatible material. This prevents liquid from causing an electrical short and prevents liquid ingress through the PCB.

In embodiment, the sensor PCB 18a also comprises an electrochemical nano/micro bubble generator to clean the sensors, as discussed in more detail below. In one example, a sensor can be used to generate nano/micro-bubbles by applying certain electrical signals and/or polarising a working electrode of the sensor in a hydrogen evolution region (HER), thus forming H₂ bubbles on the sensor electrode surface, removing contaminants as they a released.

With reference again to FIG. 2, the layered stack of electrical components 18 comprises the sensor PCB 18a, an analogue front end (AFE) PCB 18b, a control PCB 18c, a battery PCB 18d, and a radio frequency (RF) PCB 18e that are electrically connected and together form the capsule electronics system 50. The RF module 18e can be easily substituted by an alternative communications module to avail of alternative communications protocols such as ultrasound, acoustic or optical.

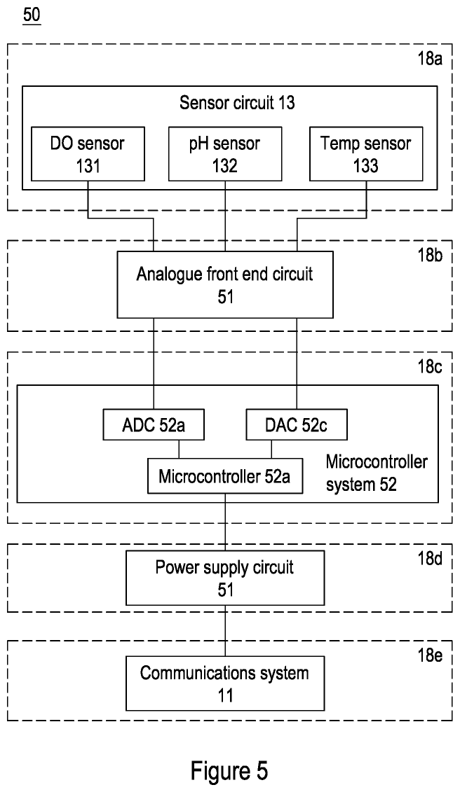

FIG. 5 shows a simplified block diagram of the capsule's electronics system 50 according to an embodiment. As described above, the sensor PCB 18a is located at the bottom of the stack 18 for coupling to the first end 12a of the sensing section 12 and comprises the sensor circuit 13 with a DO sensor 131, a pH sensor 132 and a temperature sensor 133. The AFE PCB 18b comprises an AFE circuit 51 that is configured to interface with the sensor circuit 13 and perform signal conditioning/pre-processing required to prepare sensor signals for processing at the control PCB 18c. The control PCB 18c comprises a microcontroller system 52 configured to perform control operations and data acquisition. The microcontroller system 52 comprises a microcontroller 52a, an analogue to digital convertor (ADC) 52b and a digital to analogue convertor (DAC) 52c. The ADC 52b converts the analogue signals received from the AFE circuit 51 to a digital signal suitable for the microcontroller 52a. The DAC 52c converts digital signals from the microcontroller 52a into analogue signals to be applied to the AFE 51. An example microcontroller 53 suitable for the capsule 10 is an ARM based STM32 system from ST microelectronics PLC, which has low power consumption in sleep mode. The battery PCB 18d comprises a power supply circuit 51 configured with one or more batteries to provide power to the electrical components of the stack 18. The one or more batteries can be rechargeable batteries. Optionally, the batteries may be wirelessly rechargeable to facilitate in situ charging of the capsule 10 (e.g. inductive or acoustic energy transfer). The battery PCB 18d may also comprise a power regulator circuit and/or a power management circuit. The RF PCB 18e comprises the communications systems 11 and is located at the top of the stack 18 for radiating signals out of the capsule 10. The communications systems 11 comprises a RF transceiver configured to transmit and receive RF data signals to and from an external device. Alternatively, the capsule 10 can utilise other types of communication technologies suitable for transmission through a liquid medium, such as ultrasonic or acoustic signals.

The system 50 may also comprise an accelerometer and/or an inertial measurement unit (not shown) in communication with the microcontroller 52a in order to provide to provide XYZ orientation and positional measurements in the bioreactor. The inertial data can be combined with locator signals provided by external locator device to determine a position or location of the capsule with the vessel. In one example, using the precise duration or time of flight of communication signals between the capsule and each locator, the microcontroller system 52 can use triangulation to identify the capsule location within the vessel. The external locators may be fixed at known positions inside or outside of the vessel housing the liquid body. This positional information can then be transmitted to the external device along with measurement data and used to build up data to determine the position of the capsule 10 at each measurement point for bioreactor heterogeneity evaluation. The system 50 may also comprise a memory (not shown) in communication with the microcontroller 52a configured to store the measured parameter information.

FIG. 6 shows an alternative embodiment of the capsule 10′. The capsule 10′ is the same as the capsule 10 of FIGS. 1 to 5, comprising a sensor section 12, circuitry section 14 and ballast control section 16 that connect together, but in the sensing circuit 13c is integrated into the capsule 10′ in an alternative way to allow the sensor 13 to be exchanged or replaced as needed. In this embodiment, the sensor bay is formed between the sensing section body 12′ and the ballast control section body 16′ so that the sensor 13 is accessible by removing the ballast control section body 16′. As such, the lateral side openings 15o that allow liquid to enter the sensor bay region are formed in the ballast control section body 16′, as shown. As described in more detail below, the sensor chip 13c is not directly connected to the sensor PCB 18a via wire bonding and adhesive, but is connected using an integrated circuit chip package/carrier 25a configured to hold and electrically connect to the sensor chip 13c via mechanical contact.

Element 25a is configured to be used in conjunction with elements 25d and 25c to hold the sensor chip 13c in place. Elements 25a, 25c, and 25d may be 3D printed elements. When assembled, elements 25a and 25d are positioned either side of element 25c to fix the position of element 25c. As seen in FIG. 6, element 25c contains a central aperture configured to receive the sensor chip 13c. When assembled, the sensor chip 13c is fixed in place together with element 25c. Element 25c and sensor chip 13c are effectively sandwiched between elements 25a and 25d.

Element 25d comprises conductive pogo-pins 251. The pogo-pins 251 are configured to contact the sensor chip 13c and the analogue front end (AFE) PCB (element 18a). The pogo-pins 251 facilitate electrical contact between the AFE of the PCB element 18a and the sensor chip 13c. Element 25b is a square O-ring that seals the area on the edge of the sensor chip 13c. The O-ring 25b fits inside the aperture through element 25c and surrounds the sensor chip 13c. This ensures that only the sensor electrodes on the sensor chip 13c are in contact with the liquid. Element 25c is a spacer which also acts as another sealer. Screws through elements 25d and 25a place mechanical pressure on the assembly to effectively “sandwich” the sensor 13c in place and achieve a liquid-tight seal.

The modularity of the capsule of FIG. 6 provides numerous advantages. For example, a user, or person assembling the capsule 10′ prior to use, can choose, swap, or replace sensor chip 13c depending on the desired application. A sensor chip 13c configured to monitor pH levels may be used in one embodiment of the invention, whilst a sensor chip 13c configured to monitor temperature may be used in another embodiment. Additionally, a user may disassemble the capsule 10′ to replace the sensor chip 13c without requiring an entire new capsule.

The modularity of the capsule also enables a user, or person assembling the capsule prior to use, to choose, swap or replace the capsule communications system. For example, in some embodiments an RF communications system may be used, whilst in other embodiments an acoustic, infra-red, or ultrasound communications system may be used.

As described in the preceding paragraphs, the modular design of the capsule enables a user to utilise a bespoke capsule configured to measure specific parameters and use a communications system which is suitable for a specific environment.

Due to the modular construction of the capsule 10, 10′, multiple sensing sections 12 can be employed in order to separate sensors in each sensor bay region. For example, enzymatic sensors that use hydrogen peroxide as a sensing mode can be separated to prevent cross-contamination during measurements.

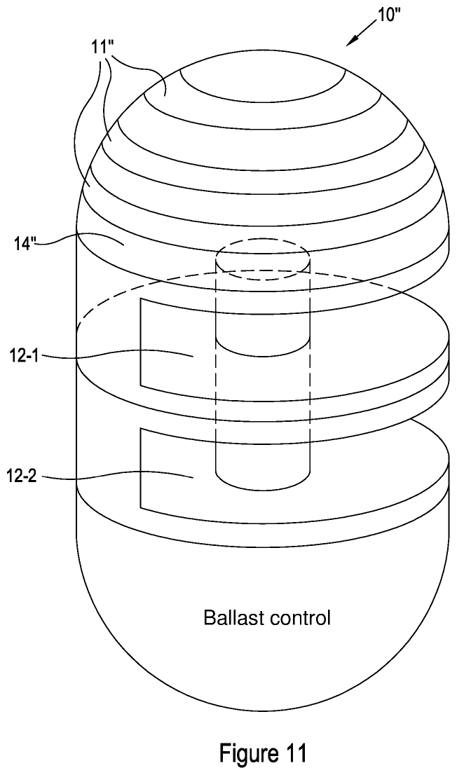

FIG. 11 shows a simplified view of a capsule 10″ comprising multiple sensor sections. The capsule 10″ comprises two sensor sections 12-1, 12-2. The separate sensor sections 12-1, 12-2 are joined together via a threaded connection, and electrical connections between the sensor sections 12-1, 12-2 can be formed using mechanical contact between complementary conductive materials on each section. For example, an electrical connection between the sections 12-1, 12-2 can be achieved using conductive ink or conductive plastic. In one example, the sensor sections 12-1, 12-2 are 3D printed, and the conductive plastic or ink incorporated into the sensing sections 12-1, 12-2 during the 3D printing process. Although the capsule 10″ is shown with two sensor sections 12-1, 12-2, three or more sensor sections could be utilised.

Where the communication system 11 generates RF signals, it comprises an antenna as is known in the art. In various embodiment, the antenna is a component that can be attached to the PCB layer 18e of the stack 18. Alternatively, the antenna may be integrated with or formed/printed on an interior surface of the circuity section body 14″ as shown in FIG. 11. This provides a larger antenna area, ability to create custom antenna patterns and thus better control the EM radiation pattern for the use application. For example, an antenna can be formed which directs the main lobe of the EM radiation pattern upwards (in use when the capsule 10 is submerged) towards a receiver device which may be located at or near the top of the liquid containing vessel (e.g. a controller antenna can be inserted into a bioreactor port which is usually on the top of traditional bioreactors).

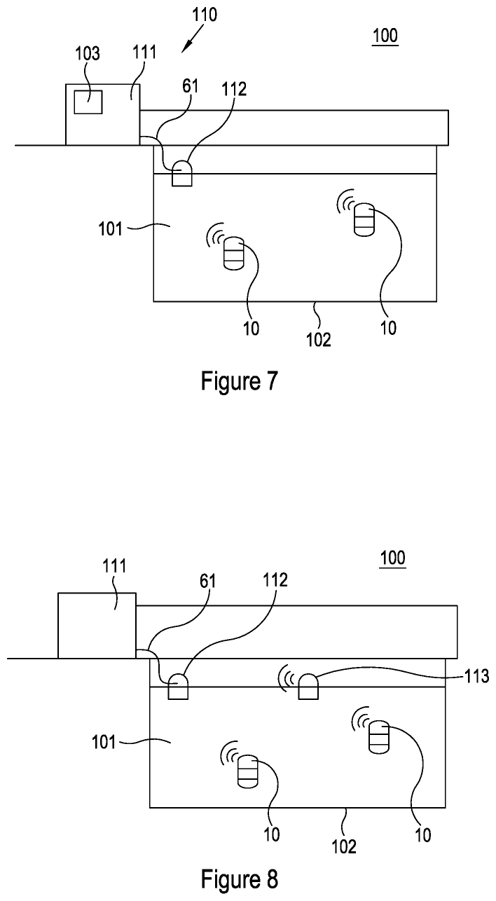

FIG. 7 shows a system 100 for monitoring one or more parameters of liquid media 101 within a bioreactor 102. The system 100 comprises one or more submersible capsules 10, 10′ for measuring one or more parameters of interest of the liquid 101 as described above, and a process control system 110 in wireless communication with the capsule 10, 10′, 10″ for controlling one or more process parameters/variables associated with the liquid 101 based on the measured parameter information.

In use, the, or each submersible capsule 10, 10′, 10″ transmits and receives data to/from the control system 110 as it floats and moves about within the liquid 101. As described above, the capsule 10, 10′, 10″ measures parameters such as pH, DO and temperature as described above, and sends the measured parameter information and optionally positional information to the controller 111. Sensor data can be sent in real time or at predefined time intervals (see below). The controller 111 can then analyse and use the sensor data to control the bioprocess and product yield, as is known in the art.

Where the system 10 comprises a plurality of capsules 10, 10′, 10′, each capsule 10, 10′, 10″ can be configured to float at a different depth in the liquid as shown to increase the volume coverage of the measurements (e.g. set using the ballast control section of each capsule 10, 10′ as described above). When combined with positional information, the control system 110 can use the capsule data to build up a 3D map of the measured liquid parameters in the bioreactor 101.

Various other signals/data can also be exchanged with the control system 110. For example, the capsule 10, 10′ can be configured to send status messages to the control system 110, periodically or in response to a request/query received from the control system 110. In particular, the capsule 10, 10′, 10″ is configured to receive commands or instructions from control system 110 to control the operation thereof. For example, the control system 110 may send instructions to increase or decrease the frequency at which parameter measurements are taken, set which parameters are measured, and/or the frequency at which data is transmitted. Instructions may be determined and sent by a user (e.g. a process engineer), using a machine learning model, based on feedback from the analysis of the bioreactor parameters, battery life of the sensor capsule 10, 10′ or various other cues. Instructions sent to the capsule 10, 10 may also include updates of the firmware and embedded software as applicable.

In one embodiment, the capsule 10, 10′ is configured to operate in a number of different modes, including but not limited to a sleep mode, and a measurement mode. The sleep mode is a low power mode in which the capsule 10, 10′ does not take any measurements and electronics components are at least partially de-activated. In the measurement mode, the capsule 10, 10′ obtains and send measurement data to the control system 110 according to one or more measurement settings which can be set/define/altered by the controller 110. Measurement settings may include measurement sample frequency, parameters measured, data exchange rate, as well as various processing operations performed by the AFE and microcontroller of the capsule 10, 10′ prior to transmission. Command signals may be used to activate and/or set an operating mode. For example, in practice the capsule 10, 10′ may spend large periods of time in sleep mode as parameters may not need to be monitored continuously. The controller 110 may send a signal to put the capsule 10, 10′ into sleep mode and wake up at various stages of the bioprocess. Commands and command sequences can be sent in response to user input to the control system 110 or pre-programmed as part of a process control sequence.

The capsule 10 may comprise default measurement and communication settings. Alternatively or additionally, these can be altered/set by the control system 110. Commands can be sent to define a measurement interval or sequence and a data exchange rate for the capsule 10, 10′. The measurement and transmit interval need not be the same. For example, where the capsule 10, 10′ comprises a memory, measurement data obtained over a period of time can be stored and accumulated in the on-board memory and sent in larger data packets to the control system 110 less frequently.

Sensor data analysis is primarily performed by the control system 110. In an embodiment, the capsule 10 is configured to perform at least some data analysis (using the microcontroller system 52) on the measured parameter information, and the communications system 11 is configured to selectively transmit measured parameter information. For example, the processor may be configured such that the capsule 10 only transmits parameter information when the measured parameter values fall outside of a predetermined range, deemed as a trigger event. The predetermined range may be stored in the memory and altered/set by the control system 110 so as to control the resolution of the measurement. Any such data analysis may be pre-programmed and/or defined by programme instructions received from the control system 110.

The control system 110 comprises a process controller 111 and a transceiver module 112 connected to the controller 111 for communicating wirelessly with the capsule 10, 10′. The controller 111 comprises one or more processors and memory for performing data analysis and process control operations. The controller 111 can be an industry standard controller such as a supervisory control and data acquisition (SCADA) system widely used in the biopharmaceutical industry, or a proprietary bioreactor controller. Existing industry standard process controllers 111 operate over a wired electrical interface with typical fixed position sensor probes. The biopharmaceutical process industry is highly regulated and slow to adopt to new technologies. As such, in the illustrated embodiment the transceiver module 112 is in wired communication with the controller 111 and is configured to connect to the process controller 111 in place of a conventional sensor probe. In this way, the transceiver module 112 acts like an adapter from wired to wireless communications enabling existing process control systems to be retrofitted and communicate with the submersible sensor capsule 10, 10′. In one example, the wired interface 61 to the controller 111 comprises a USB connection, although in principle any suitable wired communication protocol can be used. A wired connection 61 facilitates placement of the transceiver module 112 within or in proximity to the bioreactor 101, as shown. The transceiver module 112 can be a standalone device or connected to a standard bioreactor probe, as required. In other embodiments, the process controller 111 may comprise an integral wireless transceiver module for communicating with the capsule 10, 10′ (not shown).

In an embodiment, wireless communication between the capsule 10, 10′ and control system 110 is via RF signals sent to the transceiver module 112. In this case, the transceiver module 112 is an RF transceiver 112 which is configured to communicate with the RF transceiver of the capsule 10, 10′ communication system 11. Alternatively or additionally, acoustic communication can be implemented (see below). With reference again to FIGS. 2 and 6, the capsule 10, 10′ RF PCB 18e comprising the RF transceiver 11 is located at the very top of the layered stack 18 so that RF signals radiate from this point towards the destination transceiver which is, in use, above the capsule 10, 10′.

FIG. 8 shows an embodiment where the control system 110 further comprising a surface repeater 113 in wireless communication with the capsule 10, 10′ and the control system 110. In this case, the capsule 10, 10′ and controller 111 communicate via the surface repeater 113. The capsule 10, 10′, wireless transceiver 112 and surface repeater 113 form a wireless network. The surface repeater 113 is configured to float at the surface of the liquid 101, receive data/signals from the capsule 10, 10′ in the liquid 101 beneath, and relay those data/signals to the wireless transceiver 112. Additionally, the surface repeater 113 is configured to relay data/signals received from the wireless transceiver 112 to the capsule 10, 10′. The use of an intermediate surface repeater 113 reduces the communication distance between nodes in the wireless network which may be particularly useful in large bioreactors where RF signals can be significantly attenuated. In one implementation, the capsule 10, 10′, surface repeater 113 and wireless transceiver 112 are configured to communicate using RF signals. In another implementation, the capsule 10, 10′ is configured to communicate with the surface repeater 201 using acoustic signals such as ultrasound, and the surface repeater 201 is configured to communicate with the wireless transceiver 112 using RF signals. Acoustic signals are more effective for communication through a liquid medium. As both the surface repeater 113 and wireless transceiver 112 are located at or above the surface of the liquid 101, the RF signals can propagate through the air interface. The control system 110 may comprise a plurality of surface repeaters 113. In this case, each surface repeater 113 can be configured to communicate each other using RF signals through the air interface or acoustic signals along or under the surface pf the liquid 101.

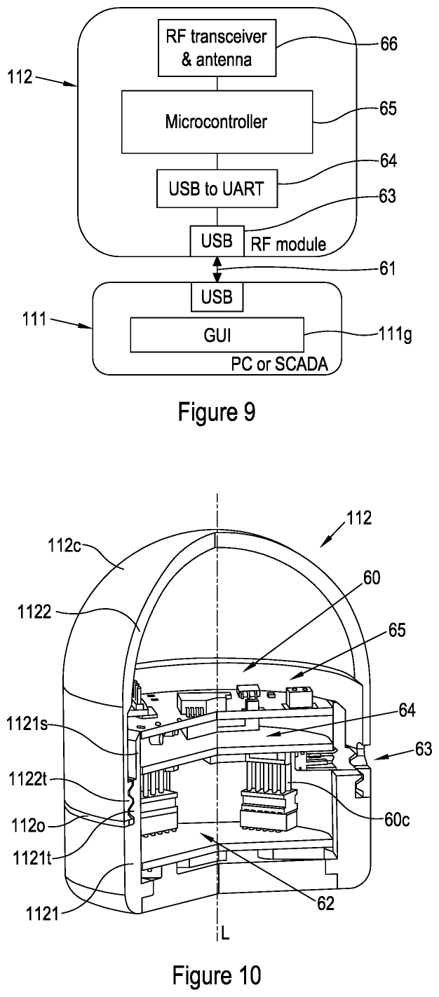

FIG. 9 shows a schematic block diagram of the transceiver module 112 and controller system 111. As described above, the controller 111 may comprise a standard SCADA controller or computing device with a graphical user interface (GUI) 111g and a USB port for connecting the transceiver module 112. The transceiver module 112 comprises a communications system 66, a microcontroller 65, a USB to UART converter 64, and a USB port 63 for connecting to the controller 111 via a wired USB connection 61. Power for the transceiver module 112 is provided by the USB port 63. The microcontroller 65 can be used to perform triangulation calculations based on locator signals received from locator devices, as discussed above. Alternatively, a battery can be provided. In an embodiment, the communication system 66 comprises an RF transceiver. The RF transceiver interfaces with a chip antenna or a larger whip antenna. Where the transceiver module 112 is attached to a standard bioreactor probe, the probe can be fitted into a bioreactor port and RF signals can be generated or received from within the bioreactor 102. This would be necessary when operating with a metal bioreactor (e.g. stainless steel) which acts as a Faraday cage preventing RF signals from penetrating through the structure, but not for non-conductive bioreactors (e.g. glass).

FIG. 10 illustrates an embodiment of the transceiver module 112 showing the construction and internal components in greater detail. The transceiver module 112 is portable. Similar to the capsule 10, 10′, the electronics of the transceiver module 112 are distributed over a plurality of PCBs arranged and connected in a layered stack 60 via connectors 60c. The stack 60 comprises an RF PCB 62, a USB port 63, a USB to UART PCB 64, and a controller PCB 65. The RF PCB 62 comprises the communications system 66 which comprises an RF transceiver circuit and chip antenna (in other embodiments a larger whip antenna can be included). The RF PCB 62 and controller PCB 65 are similar in design to that used in the capsule 10, 10, however, the embedded software for the capsule 10, 10′ and the transceiver module 112 is different. Unlike the capsule 10, 10′, the RF PCB 62 is located at the bottom of the PCB stack 60. This orientation ensures that RF signals are radiated/directed down into the bioreactor 102 if the transceiver module 112 is positioned at the top of the bioreactor 102. The external controller 111 interfaces with the transceiver module 112 via the USB port 63.

The transceiver module 112 comprises a substantially rigid hollow casing 112c containing the transceiver electronics. Like the capsule 10, 10′, the transceiver module 112 has a modular construction comprising a base section 1121 and a cover section 1122 that connect together along a longitudinal axis L using a thread 1121t, 1122t and o-ring 112oconnection mechanism to form sealed interior space within the assembled casing 112c. The base section 1121 comprises a threaded portion 1121t configured to engage corresponding threaded portion 1122t of the cover portion 1222, and a sleeve portion 1211r configured to receive the layered stack 60 of electrical components. The bottom PCB layer (the RF PCB 62) attaches to the base section 1121. The rest of the stack 60 connects to the RF PCB 62 via electrical connectors 60c which may also serve to hold it in place.

The dimensions of all the capsule, transceiver module 112 and PCB boards and stacks 18. 60 can be the same to enable sections, electronics assemblies and components to be swapped in and out of different embodiments in order to grow a system.

In the embodiment shown, the sleeve 1121a and the layered stack 60 comprise complementary geometric alignment features such as lateral side flats (not shown) to aid proper alignment of the layered stack 60 and connectors 60c a upon insertion. In various embodiments, the sleeve 1121s may also be configured to grip/hold the stack 60 in position by frictional and/or mechanical engagement to prevent unintentional disconnection of the PCB layers. Additionally or alternatively, when assembled, the cover portion 1222 is configured to apply a pressure or retaining force on the top of the layered stack 60 to hold it in place against the base section 1121.

The section bodies 1121, 1122 that form the transceiver module 112 casing 112c are formed of or comprise a substantially rigid plastic material (e.g. using a moulding process) to provide durability and resistance to chemical and physical damage within the bioreactor environment. In an embodiment, the plastic material is or comprises a thermoplastic, such as polycarbonate, polypropylene, polyethylene, cyclic olefin copolymer (COC), together with silicone as applicable.

In embodiments where the capsule 10, 10′ comprises an accelerometer, and optionally a triangulation module linked to external locator devices as detailed below, for providing positional data, the control system 100 may also comprise an accelerometer 103 to provide fixed reference XYZ positional data (see FIG. 7). This capsule and reference accelerometer data can be used by the control system 110 to determine the position of the capsule 10, 10′ at each measurement point and generate a 3D map of the bioreactor environment.

In some embodiments, the transceiver module 112 is configured to float on or within the liquid body at a certain depth at a fixed position, or within a certain region, as shown in FIGS. 7 and 8.

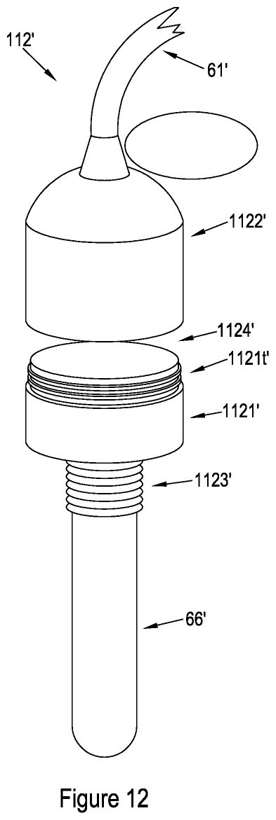

FIG. 12 shows an example transceiver module 112′ in accordance with another embodiment. The transceiver module 112′ comprises, or is attachable to, a probe configured is configured to be inserted into a port of a bioreactor (or liquid containing vessel) which houses the liquid body. In this embodiment, the antenna 66′ of the transceiver 112′ is a long probe-like form factor. The antenna 66′comprises screw threads 1123′ which screw into the bioreactor port and fix the position of the transceiver 112′. Such an arrangement positions the antenna inside the stainless-steel tank (which acts as a Faraday cage), thereby allowing the RF signals from the capsule 10, 10′ to be received by the transceiver module 112′. The RF signals can then be translated to a USB signal for processing outside the bioreactor 102. A USB cable 61′ is used connect the transceiver module 112′ to a main SCADA controller so that the signals can be processed and/or analysed.

In FIG. 12, the electrical components are not shown, but they are positioned between the base section 1121′ and the cover portion 1122′, similar to the embodiment of FIG. 10. The base section 1121′ and the cover portion 1122′join via threaded connection using threaded section 1112t′.

In other embodiments, single use bioreactors may be used. For single use plastic bag bioreactors, transceiver module 112 can be fixed to the side of the bag as RF signals will pass through the plastic bag easily.

The control system 110 may also comprises one or more locator devices (not shown). The locators send locating signals to the capsule 10, 10′ and the controller 111 via the transceiver module 112. The locating signals contain information indicating where the signals originated, and when they were first sent. When the capsule 10, 10′ receives the locating signal, the time the locating signal was received is recorded. The capsule 10, 10′ microcontroller is configured to determine a location or position of the capsule 10 within the liquid body based on the locator signals using triangulation or multilateration (optionally using additional sensor data from the accelerometer). The capsule communications system then sends the determined position information to the transceiver module 112 along with the measured parameter information. In this case, the transmitted parameter information can includes the determined capsule position at the parameter measurement/sample time allowing the controller 111 to perform further analysis and generate 3D maps of the parameter values throughout the bioreactor 102.

Alternatively, the transceiver module 112 can perform the triangulation calculation based on information in the locator signals relayed from the capsule 10. The capsule 10, 10′ generates location information at the time each parameter measurement is taken or sample. The location information, in some embodiments, is the time at which previous locating signals were received, together with the information in that locating signal (i.e. which locator the signal originated from, the position of the locator, the time at which the locating signal was sent, etc.). This location information is tagged to the measurement sample. The location information is then sent to the transceiver module 112 together with the parameter information. A microcontroller of the transceiver module 112 can then work out the position of the capsule 10 using a triangulation calculation based on the location information in the received data. In this way, AI can be performed at the edge and data scrubbed before sending it to the main control (e.g. SCADA) system 110. The control system 110 software can perform further analysis afterwards.

In an embodiment, the system 100 further comprises a charging or docking station configured to wirelessly charge the capsule battery in situ (not shown). The charging station is mountable at least partially within the liquid and comprises a means to capture the capsule 10, 10′ within the liquid and move the capsule 10, 10′ into proximity with the charging station for power transfer. The charging station can be configured to wirelessly charge the capsule battery using inductive coupling, magnetic coupling or acoustic coupling, as is known the art.

The immersion of an electrochemical sensor, such as those which be used in the capsule of the present invention, in a liquid media leads to bio-fouling due to the adsorption of proteins on the surface of the sensor, which in turn leads to degradation in sensing capability. Electrical signals can be used to generate nano-bubbles at the surface of a sensor electrode to perform in-situ cleaning of the sensor's surface. The nano-bubbles push the contaminants (such as proteins) away from the sensor surface prior to a sensing operation.

Sensor cleaning follows a three-step process involving a voltage sequence that generates the nano/micro bubbles, performs double layer capacitance management and then a sensor measurement. The first step is generating the nano-bubbles by polarization of a working electrode in the Hydrogen evolution region (HER). For example, the capsule 10 may use enzymatic sensors which are formed by entrapping an enzyme in a polymer that is deposited on an electrode through a process of electro-polymerization. The nano-bubble produced at the surface of the sensor is forced from the surface of the electrode through the polymer.

The second step in the process involves polarizing the working electrode to a higher potential. This causes dissipation of the capacitive charge built up during the first step. This is necessary since the capacitive charge would generate a current that would dominate measurement during the third step.

The third step involves the polarization of the working electrode to a voltage that allows detection of the analyte. This three-step cycle can be repeated multiple times to achieve satisfactory measurements.

From reading the present disclosure, other variations and modifications will be apparent to the skilled person. Such variations and modifications may involve equivalent and other features which are already known in the art of garments incorporating electronic capabilities, and which may be used instead of, or in addition to, features already described herein

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The applicant hereby gives notice that new claims may be formulated to such features and/or combinations of such features during the prosecution of the present application or of any further application derived therefrom.

For the sake of completeness, it is also stated that the term “comprising” does not exclude other elements or steps, the term “a” or “an” does not exclude a plurality, a single processor or other unit may fulfil the functions of several means recited in the claims and any reference signs in the claims shall not be construed as limiting the scope of the claims.

1. A submersible sensor capsule for monitoring one or more parameters of a liquid body in which the capsule is submersed, the capsule comprising:
one or more sensors configured to measure one or more parameters of the liquid in which the capsule is submersed;
an electronics assembly, the electronics assembly comprising:
a sensor circuit in electrical communication with the one or more sensors; and
a wireless communications system configured to transmit measured parameter information to an external device;
wherein the capsule is configured to isolate the electronics assembly from the liquid and permit liquid to contact at least a portion of the one or more sensors; and
wherein the capsule is configured to float at a depth within the liquid body.

2. The capsule of claim 1, wherein the communications system is configured to transmit measured parameter information to an external device periodically or according to a predefined schedule.

3. The capsule of claim 1, wherein the communications system is configured to selectively transmit measured parameter information to an external device in response to a trigger signal received from the external device and/or in response to a detected trigger event; and/or
wherein a trigger event comprises one or more of: a threshold change in one or more of the measured parameters, a threshold rate of change in one or more of the measured parameters, and a detection of a parameter.

4. The capsule of claim 2, wherein the communication system is configured to transmit the measured parameter information to an external device periodically at a transmit frequency, and wherein the communication system is configured to alter the transmit frequency in response to one or more of: a change in the operating mode of the capsule, a trigger signal received from the external device, and a detected trigger event.

5. (canceled)

6. The capsule of claim 3, wherein the electronics assembly comprises a processing circuit configured to analyse the measured parameter information and detect a trigger event; and optionally, wherein the processing circuit is configured to sample the output of the one or more sensors at a measurement frequency, and wherein the processing circuit is configured to alter the measurement frequency in response to one or more of: a change in the operating mode of the capsule, a trigger signal received from the external device, and a detected trigger event.

7. (canceled)

8. The capsule of claim 1, wherein the capsule comprises a sensor bay region in which the one or more sensors are positioned and/or mounted, and one or more side openings to allow liquid to enter the sensor bay region to contact the one or more sensors; and optionally, further comprising a circuitry section and a sensing section removably connected to the circuitry section along a longitudinal axis, wherein the connected circuitry section and sensing section define an enclosed space within the circuitry section housing the electronics assembly, and the sensing section at least partially defines the sensor bay region; and wherein the enclosed space of the circuitry section is fluidly isolated from liquid in the sensor bay region; and optionally or preferably, wherein the sensing section comprises the one or more side openings; and/or
wherein the capsule comprises a fluidic actuator configured to convey liquid through the openings into and/or through the sensor bay region, and optionally or preferably, wherein the fluidic actuator comprises a micropump; and or
wherein the sensing section comprises an aperture in a longitudinal end adjacent the circuitry section and the one or more sensors are positioned or mounted over or within the aperture to permit liquid in the sensor bay region to contact a surface of the one or more sensors and electrical connection to the sensing circuit in the circuitry section; and optionally or preferably,
wherein the one or more sensors are positioned so as to seal the aperture to prevent liquid entering the circuitry section.

9. (canceled)

10. (canceled)

11. (canceled)

12. The capsule of claim 1, wherein the capsule comprises a ballast control section configured to control the depth at which the capsule floats within the liquid and/or the orientation of the capsule within the liquid; and optionally, wherein the circuitry section is removably connected to a first end of the sensing section and the ballast control section is removably connected to a second, opposing end of the sensing section.

13. (canceled)

14. The capsule of claim 1, wherein the communications system is further configured to receive signals from an external device; and, optionally or preferably, wherein the signals comprise control signals with instructions to set or change the measurement frequency or the transmit frequency; and/or
wherein the components of the electronics assembly are arranged in a layered stack, and optionally or preferably, wherein the electronics assembly further comprises one or more of: a battery, a control system, a memory, a processor, an analogue to digital converter, a digital to analogue converter, and an analogue front-end adapter; and optionally,
wherein the communications system is disposed at a first end of the layered stack and the one or more sensors are disposed at a second of the layered stack, opposite the first; and optionally or preferably wherein the capsule is configured, when submersed in liquid, to be oriented such that the first end is above the second end.

15. (canceled)

16. (canceled)

17. The capsule of claim 1, wherein the one or more sensors are configured to measure one or more of: pH level, temperature, glucose, lactate, and dissolved oxygen; and/or
wherein the capsule comprises one or more positional sensors configured to generate sensor data containing position information of the capsule within the liquid, and optionally wherein the capsule communications system is configured to send the position information to the external device; and, optionally or preferably,
wherein the one or more positional sensors comprise one or more of: an accelerometer, gyroscope and an inertial measurement unit; and/or
wherein the electronics assembly comprises a processing circuit configured to determine a location or position of the capsule within the liquid body based on signals received from a plurality of external devices, and wherein the capsule communications system is configured to send the determined position information to the external device, optionally or preferably using triangulation or multilateration; and further optionally or preferably, wherein the transmitted parameter information includes the capsule position at the parameter measurement time.

18. (canceled)

19. (canceled)

20. The capsule of claim 1, wherein the capsule comprises a plurality of sensors for measuring different parameters of the liquid; and, optionally,
wherein the capsule comprises a plurality of sensing sections, each sensing section at least partially defining a sensor bay region in which one or more sensors are positioned and/or mounted.

21. (canceled)

22. The capsule of claim 1, wherein the communications system is configured to generate radio frequency signals, acoustic signals, or ultrasound signals; and/or
wherein the capsule has a modular construction comprising a plurality of interchangeable sections, including a circuitry section that houses the electronics assembly, a sensing section at least partially defining a sensor bay region in which one or more sensors are positioned and/or mounted to contact liquid, and optionally a ballast control section configured to control the depth at which the capsule floats within the liquid and/or the orientation of the capsule within the liquid; and/or.
further comprising a micro or nano-bubble generator configured to clean a surface of the one or more sensors in contact with the liquid; and optionally or preferably, wherein the micro or nano-bubble generator is configured to direct generated micro or nano-bubbles over the surface of the sensor.

23. (canceled)

24. (canceled)

25. A system for monitoring one or more parameters of a liquid, the system comprising:
a submersible sensor capsule as defined in claim 1; and
a transceiver module connectable to an external device;
wherein the transceiver module is configured to receive measured parameter information transmitted by the capsule and provide the measured parameter information to a connected external device.

26. The system of claim 25, wherein the transceiver module comprises, or is attachable to, a probe configured to be disposed in the liquid to receive the measured parameter information from the capsule.

27. The system of claim 25, wherein the capsule comprises one or more positional sensors configured to produce sensor data containing positional information of the capsule within the liquid, and the capsule's communications system is configured to send the sensor data to the transceiver module together with the measured parameter information; and/or
further comprising one or more locator devices configured to send locating signals to the capsule,
wherein the electronics assembly of the capsule comprises a processing circuit configured to determine a location or position of the capsule relative to the one or more locators and/or the external device based on the locating signals received, optionally or preferably using triangulation or multilateration, and
wherein the capsule's communications system is configured to send the determined position information to the transceiver module together with the measured parameter information; and optionally or preferably, wherein the transmitted parameter information includes the capsule position at the parameter measurement time.

28. (canceled)

29. The system of claim 27, wherein the capsule processing circuit is configured to determine a location or position of the capsule relative to the one or more locators based on the received locating signals and the sensor data; and/or
wherein the system comprises the external device, and the external device is configured to generate a map indicating measured parameter values at one or more locations in the liquid based on the received parameter information and capsule location information, and, optionally, wherein the external device is configured to send instructions to the capsule via the transceiver module.

30. (canceled)

31. The system of claim 25, wherein the system comprises the external device, and the external device is or comprises a controller configured to control at least one of the parameters of the liquid based at least in part on the measured parameter information, and optionally or preferably, wherein the system is a bioreactor control system.

32. (canceled)

33. The system of claim 25, further comprising one or more surface repeaters configured to receive signals transmitted by the capsule's communications system and relay the signals to the transceiver module, and optionally or preferably, wherein the surface repeaters are configured to float or be positioned at the surface of the liquid; and, optionally,
wherein the capsule communications system is configured to send ultrasound signals to the one or more surface repeaters; and/or
wherein the transceiver module comprises a radio frequency transceiver, and the surface repeaters are configured to send radio frequency signals to the transceiver module.

34. (canceled)

35. (canceled)

36. The system of claim 25, wherein the capsule comprises a rechargeable battery, and the system comprises a charging station configured to charge the capsule battery in situ, and optionally or preferably, wherein the charging station is configured to wirelessly charge the capsule battery using inductive coupling, magnetic coupling or acoustic coupling; and optionally,
wherein the charging station is mountable at least partially within the liquid and comprises a means to capture the capsule within the liquid and move the capsule into proximity with the charging station for power transfer.

37. (canceled)

38. The system of claim 25, wherein the system comprises a plurality of submersible sensor capsules; and optionally, as defined in claim 1
wherein each of the plurality of sensor capsules is configured to float at a different depth within the liquid; and/or wherein each capsule is configured to float in a distinct region of the liquid.

39. (canceled)

40. A submersible sensor capsule for monitoring one or more parameters of a liquid body in which the capsule is submersed, the capsule comprising:
one or more sensors configured to measure one or more parameters of the liquid;
a wireless communications system configured to transmit measured parameter information to an external device; and
micro or nano-bubble generator configured to remove contaminants from a surface of the one or more sensors;
wherein the capsule is configured to float at a depth within the liquid body, and optionally or preferably, wherein the micro or nano-bubble generator is configured to direct generated micro or nano-bubbles over the surface of the sensor.
